(19) United States
(12) Patent Application Publication
(10) Pub. No.: US 2020/0048184 A1
(43) Pub. Date: Feb. 13, 2020

(54) METHOD AND APPARATUS FOR PRODUCING ALKYL NITRITE

(71) Applicant: UBE INDUSTRIES, LTD., Ube-shi, Yamaguchi (JP)

(72) Inventors: Hirofumi II, Ube-shi (JP); Naoki NEGORO, Ube-shi (JP); Akira HINO, Ube-shi (JP); Yoshikatsu NISHIDA, Ube-shi (JP)

(73) Assignee: UBE INDUSTRIES, LTD., Ube-shi, Yamaguchi (JP)

(21) Appl. No.: 16/498,917

(22) PCT Filed: Jan. 31, 2018
(86) PCT No.: PCT/JP2018/003218

§ 371 Date: Sep. 27, 2019

(30) Foreign Application Priority Data
Mar. 29, 2017 (JP) 2017-065796
Sep. 27, 2017 (JP) 2017-187121

(51) Int. Cl. C07C 201/02; C07C 203/02

(52) U.S. Cl. CPC C07C 201/02; C07C 203/02

(57) ABSTRACT

A method for producing an alkyl nitrite by bringing an aqueous solution containing nitric acid and an alkanol into contact with a gas including nitrogen monoxide and thereby producing an alkyl nitrite, in which the reaction temperature is 60° C. to 100° C., is provided.

### TECHNICAL FIELD

The present disclosure relates to a production method and a production apparatus for producing an alkyl nitrite.

### BACKGROUND ART

As a method for producing an alkyl nitrite, methods of reacting nitrogen monoxide, oxygen, and an alkanol are known (for example, Patent Literature 1 and Patent Literature 2).

Also, regarding a method for continuously producing dimethyl carbonate from carbon monoxide and methyl nitrite, a method of causing nitrogen monoxide produced to react with oxygen and methanol and thereby regenerating methyl nitrite is known. In such a method, a technology of supplying nitric acid as a nitrogen source together with the gas is known (for example, Patent Literature 3).

A method of bringing an aqueous solution containing nitric acid and an alkanol into contact with nitrogen monoxide gas and producing an alkyl nitrite is also known (see, for example, Patent Literature 4 and Patent Literature 5).

In a method of bringing nitrogen monoxide, nitric acid, and an alcohol into contact with one another in a reactor for nitric acid conversion and producing a nitrous acid ester, a technology of concentrating the reaction liquid in the reactor for nitric acid conversion using a nitric acid concentrating column, and returning the reaction liquid thus concentrated to the reactor for nitric acid conversion, is known. In this technology, the nitric acid concentration in the reactor for nitric acid conversion can be increased (for example, Patent Literature 6).

### CITATION LIST

### Patent Literature

Patent Literature 1: Japanese Unexamined Patent Publication No. H11-189570

Patent Literature 2: Japanese Unexamined Patent Publication No. H6-298706

Patent Literature 3: Japanese Unexamined Patent Publication No. 116-25104

Patent Literature 4: Japanese Patent No. 4026521

Patent Literature 5: Japanese Patent No. 4134777

Patent Literature 6: Japanese Patent No. 6070695

### SUMMARY OF INVENTION

### Technical Problem

In regard to the production methods of Patent Literature 1 and Patent Literature 2, since a significant amount of nitric acid is produced as a byproduct, the loss of nitrogen component increases. Therefore, there is a demand for a method of making it possible to effectively reuse the nitric acid thus produced as a byproduct. Furthermore, in Patent Literature 1, the column bottom liquid (containing nitric acid) of the reaction column for alkyl nitrite regeneration is pulled out, and then while this is cooled and circulated into the reaction column, alkyl nitrites are produced. Therefore, it has been difficult to efficiently produce methyl nitrite from nitric acid, nitrogen monoxide, and methanol.

In regard to the production method of Patent Literature 3, the decomposition reaction for nitric acid is not efficient, and the applicable temperature range is also limited. Furthermore, it is difficult to efficiently produce methyl nitrite from nitric acid, nitrogen monoxide, and methanol.

As is the case of the production methods of Patent Literature 4 and Patent Literature 5, even if a method of converting nitric acid into a nitrous acid ester using nitrogen monoxide or carbon monoxide is used, a significant amount of nitric acid is included in the effluent discharged from the reactor for nitric acid conversion. Therefore, it has been difficult to lower the nitric acid concentration in the effluent.

In Patent Literature 6, the reaction liquid in the reactor for nitric acid conversion is concentrated using a nitric acid concentrating column, and the reaction liquid thus concentrated is returned to the reactor for nitric acid conversion. Thereby, the nitric acid concentration in the reactor for nitric acid conversion is increased, and effective utilization of nitric acid in the reaction liquid is attempted.

However, in the case of using a concentrating column, the cost of equipment is needed due to additional establishment of concentrating columns, and energy for the concentrating operation is also needed. On the other hand, in a case in which a concentrating column is not used, since the amount of effluent after neutralization of nitric acid is increased, the environmental burden tends to increase. Under such circumstances, there is a demand for establishing a technology by which the nitric acid concentration in the effluent can be sufficiently decreased even without using a concentrating column.

Thus, according to an aspect, it is an object of the present invention to provide an industrially suitable method for producing an alkyl nitrite, by which an alkyl nitrite can be efficiently produced by effectively utilizing nitric acid. According to another aspect, it is an object of the present invention to provide an industrially suitable apparatus for producing an alkyl nitrite, by which an alkyl nitrite can be efficiently produced by effectively utilizing nitric acid.

### Solution to Problem

According to an aspect, the present invention provides a method for producing an alkyl nitrite, the method including bringing an aqueous solution containing nitric acid and an alkanol into contact with a gas including nitrogen monoxide and thereby producing an alkyl nitrite, in which the reaction temperature is 60° C. to 100° C.

According to the production method described above, a reaction for producing an alkyl nitrite from nitric acid, an alkanol, and nitrogen monoxide proceeds sufficiently. Therefore, an alkyl nitrite can be efficiently produced by effectively utilizing nitric acid. Since this production method proceeds until nitric acid reaches a low concentration, nitric acid can be effectively utilized even without providing a nitric acid concentrating column. Therefore, it is said that the above-mentioned production method is an industrially suitable production method.

The concentration of the alkanol may be 50% by weight to 80 by weight, or may be 60% by weight to 80% by weight, with respect to the total amount of the aqueous solution. In the above-mentioned production methods, the nitric acid concentration in a reaction liquid obtainable by bringing the aqueous solution into contact with the gas may be decreased to 1% by weight or less. The alkanol may include methanol. The concentration of nitric acid in the aqueous solution may be 1% by weight to 20% by weight with respect to the total amount of the aqueous solution.

The production method described above may have a first reaction step of producing an alkyl nitrite at a reaction temperature of 60° C. to 80° C.; and a second reaction step of producing an alkyl nitrite at a reaction temperature of 80° C. to 100° C. The superficial gas velocity in the first reaction step may be 20 mm/second to 100 mm/second, and the superficial gas velocity in the second reaction step may be 1 mm/second to 20 mm/second. The production method described above may produce the alkyl nitrite using a reactor that is compartmented into two or more chambers along the flow direction of the aqueous solution. Meanwhile, the phrase “being compartmented along the flow direction” means that reaction tanks in the reactor are arranged in series.

The reactor may have a first reaction unit that is operated in a temperature range of 60° C. to 80° C., and a second reaction unit that is disposed on the downstream side of the first reaction unit and operated in a temperature range of 80° C. to 100° C. The superficial gas velocity at the first reaction unit may be 20 mm/second to 100 mm/second, and the superficial gas velocity at the second reaction unit may be 1 mm/second to 20 mm/second. It is also acceptable that the first reaction unit is located on the most upstream side of the reactor, and the second reaction unit is located on the most downstream side of the reactor. The use amount of a catalyst containing a transition metal with respect to the aqueous solution may be less than 0.1% by weight in terms of the transition metal. It is preferable to perform the first reaction step at the first reaction unit, and it is preferable to perform the second reaction step at the second reaction unit.

According to another aspect, the present invention provides an apparatus for producing an alkyl nitrite, the apparatus including a reactor for bringing an aqueous solution containing nitric acid and an alkanol into contact with a gas including nitrogen monoxide and thereby producing an alkyl nitrite, in which the reaction temperature in the reactor is 60° C. to 100° C.

According to the production apparatus described above, the reaction for producing an alkyl nitrite from nitric acid, an alkanol, and nitrogen monoxide can be sufficiently carried out. Therefore, an alkyl nitrite can be efficiently produced by effectively utilizing nitric acid. Since this production apparatus can produce an alkyl nitrite until nitric acid reaches a low concentration in the reactor, nitric acid can be effectively utilized even without providing a nitric acid concentrating column. Therefore, it is said that the above-mentioned production apparatus is an industrially suitable production apparatus.

The reactor may have a plurality of reaction units compartmented along the flow direction of the aqueous solution. The reactor may have a first reaction unit, and a second reaction unit that is located on the downstream side of the first reaction unit and produces the alkyl nitrite at a reaction temperature higher than the reaction temperature of the first reaction unit. In the reactor, the nitric acid concentration in the reaction liquid may be decreased to 1% by weight or less.

### Advantageous Effects of Invention

According to the present disclosure, an industrially suitable production method and an industrially suitable production apparatus for an alkyl nitrite, by which an alkyl nitrite can be efficiently produced by effectively utilizing nitric acid, can be provided. This production method and production apparatus can sufficiently lower the nitric acid concentration in the effluent, even without using a concentrating column. Therefore, the cost of equipment can be reduced.

### BRIEF DESCRIPTION OF DRAWINGS

FIG. 1 is a schematic diagram illustrating an example of a reactor.

### DESCRIPTION OF EMBODIMENTS

In the following description, several embodiments of the present invention will be explained with reference to the drawings according to the cases. However, the following embodiments are merely examples for explaining the present invention and are not intended to limit the present invention to the following matters.

The method for producing an alkyl nitrite of the present embodiment has a reaction step of bringing an aqueous solution containing nitric acid and an alkanol into contact with a gas including nitrogen monoxide, and producing an alkyl nitrite. Alkyl nitrites are compounds useful for various oxidation processes (production of a dialkyl oxalate, a dialkyl carbonate, and the like).

In regard to the aqueous solution containing nitric acid and an alkanol (hereinafter, also referred to as nitric acid-containing aqueous solution), which is used as a raw material, the nitric acid concentration is not particularly limited and may be a high concentration or a low concentration. From the viewpoint of further increasing the industrial efficiency, the nitric acid concentration may be 1% by weight to 60% by weight, may be 1% by weight to 20% by weight, or may be 1% to 10% by weight, with respect to the total amount of the aqueous solution.

Examples of the alkanol include alkanols having 1 to 3 carbon atoms (methanol, ethanol, n-propanol, and i-propanol). From the viewpoint of industrially suitably purifying an alkyl nitrite, it is preferable that the alkanol includes methanol, and it is more preferable that the alkanol is composed only of methanol. However, in a case in which the alcohol is composed only of methanol, the alcohol may include unavoidable impurities.

The alkanol is included in an aqueous solution. The alkanol may be uniformly dissolved or mixed in an aqueous solution. The concentration of the alkanol is not particularly limited and may be adjusted appropriately according to the reaction conditions or apparatus. From the viewpoint of further increasing the industrial efficiency, the concentration of the alkanol may be 50% by weight or more, 60% by weight or more, or 65% by weight or more, with respect to the total amount of the aqueous solution. The concentration of the alkanol in the aqueous solution may be 5% by weight to 90% by weight, may be 50% by weight to 90% by weight, may be 50% by weight to 85% by weight, may be 50% by weight to 80% by weight, may be 60% by weight to 80% by weight, or may be 60% by weight to 75% by weight.

When the aqueous solution is brought into contact with a solution having a high nitric acid concentration while the, alkanol concentration is high, there is a risk that an alkyl nitrate may be produced. In the present embodiment, production of the alkyl nitrate can be suppressed by adjusting the concentrations of the alkanol and nitric acid to the above-mentioned range. The alkanol may be collected by distillation or the like from the reaction liquid after the reaction step and may be reused in the production method of the present embodiment.

The gas including nitrogen monoxide to be used in the present embodiment may be pure nitrogen monoxide gas, or may be a mixed gas diluted with an inert gas such as nitrogen. Such a gas is supplied to the reaction system. It is preferable that the mixed gas contains nitrogen monoxide at 4% by volume or more. The unit “percent (%) by volume” as used in the present disclosure is a volume proportion in the standard state (0° C., 1 atm). The mixed gas may further include gas components that do not interrupt the progress of the reaction (carbon monoxide, carbon dioxide, alcohol vapor, and the like).

In order to produce an alkyl nitrite efficiently, it is preferable that the gas including nitrogen monoxide does contain nitrogen oxides that are produced as a result of the presence of molecular oxygen in the gas. It is preferable that the gas including nitrogen monoxide does not contain, for example, nitrogen dioxide, dinitrogen trioxide, dinitrogen tetraoxide, and molecular oxygen. In addition, nitrogen monoxide may be used at a proportion of 1 mol to 50 mol, 1.5 mol to 20 mol, or 2 to 10 mol, with respect to 1 mol of nitric acid.

The feed amount (supply amount) of the gas including nitrogen monoxide may be 0.1 NL/h to 500 NL/h per 1 L of the capacity of the reactor, or may be 0.3 NL/h to 300 NL/h. Meanwhile, the feed amount of the gas including nitrogen monoxide may be appropriately regulated according to the shape of the reactor, the purity of nitrogen monoxide in the gas, the reaction temperature, stirring speed, and the like.

In the reaction step, an aqueous solution containing nitric acid and an alkanol is brought into contact with a gas including nitrogen monoxide. Thereby, nitric acid reacts with nitrogen monoxide and the alkanol, and thus an alkyl nitrite is produced. From the viewpoint of efficiently lowering the nitric acid concentration in the aqueous solution, the reaction temperature may be 0° C. to 200° C., may be 20° C. to 100° C., or may be 60° C. to 100° C. When the reaction temperature becomes too high, methyl nitrite further reacts, and methyl nitrate may be produced. In the present embodiment, as described above, even if the reaction temperature is made higher, the production amount of methyl nitrate can be suppressed.

The pressure (reaction pressure) in the reaction step may be from atmospheric pressure to 20 MPaG, may be from atmospheric pressure to 3 MPaG, or may be 0.2 MPaG to 1 MPaG. In the present embodiment, the reaction may be carried out under pressure as such. It is preferable that the aqueous solution containing nitric acid and an alkanol to be used in the reaction step does not contain nitrogen oxides that are produced due to the presence of molecular oxygen in the gas including nitrogen monoxide.

In the present embodiment, a transition metal compound may be incorporated as a catalyst into the aqueous solution. Examples of the transition metal compound include nitrates, oxides, and the like of the metals of Groups 3 to 11 of the Periodic Table. Specific examples include, as nitrates of the metals of Group 8, ferric nitrate, nickel nitrate, and cobalt nitrate. Examples of the nitrates of the metals of Group 11 (1B metals) include cupric nitrate. Examples of other transition metal compounds include oxides of manganese, cerium, zirconium, cobalt, molybdenum, and vanadium. The amount of the catalyst may be 20% by weight or less in terms of metals, may be 10% by weight or less, or may be 0.1% by weight to 10% by weight, in the aqueous solution to be used as a raw material.

In the present embodiment, an alkyl nitrite can be produced without incorporating a transition metal in an amount sufficient for producing an alkyl nitrite by bringing an aqueous solution containing nitric acid and an alkanol into contact with a gas including nitrogen monoxide. An amount sufficient for producing an alkyl nitrite is 0.1% by weight or more in terms of metal in the aqueous solution. The transition metal compound may be supported on a material such as activated carbon, carbon black, an organic polymer, zeolite, a mesoporous silicate, alumina, silica, clay, diatomaceous earth, and pumice. However, it is not essential to use a catalyst, and the aqueous solution may not include a catalyst.

When an alkyl nitrite is produced without incorporating a catalyst in an amount sufficient for producing an alkyl nitrite, the material cost and the number of processes necessary for production can be reduced. Therefore, a more industrially suitable method for producing an alkyl nitrite can be established.

In the reaction step, the reaction proceeds in the liquid phase. The reaction step may be batch type or may be continuous type. This reaction step can be carried out in a reactor. In the reaction step, it is preferable that the reaction is carried out while the aqueous solution is stirred. The reactor may be, for example, a stirring tank having a stirrer, a gas-liquid mixing reaction tank, a packed column, a plate column, or a bubble column. The type of the reactor is not limited to these, and generally known reactors other than the above-mentioned reactors may also be used. By using such a reactor, nitric acid, an alkanol, and nitrogen monoxide can be caused to react efficiently.

In the reaction step, an aqueous solution containing nitric acid and an alkanol may be supplied to the reactor, and the aqueous solution may be stirred at normal pressure or under pressure while a gas including nitrogen monoxide is caused to flow into the aqueous solution. Alternatively, nitrogen monoxide gas may be injected into the reactor, and the aqueous solution may be stirred under pressure. At this time, it is preferable that nitrogen oxides produced as molecular oxygen is present in the nitrogen monoxide gas is not included in the gas including nitrogen monoxide and is not supplied to the reaction system. It is preferable that nitrogen oxides are also not included in the aqueous solution containing nitric acid and an alkanol, which is supplied to the reactor.

The reaction step may be divided into a plurality of stages. The temperatures at the respective steps may be set arbitrarily within the above-mentioned temperature ranges for the reaction step. From the viewpoint of efficiently reducing the nitric acid concentration in the aqueous solution, the reaction step may include, for example, a first reaction step of producing an alky nitrite at a reaction temperature of 60° C. to 80° C., and a second reaction step of producing an alkyl nitrite at a reaction temperature of 80° C. to 100° C. The reaction temperature of the first reaction step may be 65° C. to 75° C. The reaction temperature of the second reaction step may be 85° C. to 95° C.

When the reaction step is divided into a plurality of stages, it is not necessarily essential that the reactor is compartmented by piping or the like, and it is desirable when regions at the above-mentioned temperature ranges are present in the same reactor. It is still acceptable to have reaction steps at different temperature ranges before and after the first reaction step and the second reaction step. It is preferable that the first reaction step is carried out in a reaction unit on the most upstream side, and it is preferable that the second reaction step is carried out in a reaction unit in the most downstream side. Meanwhile, when the reaction step is divided in a plurality of stages, the terms “upstream” and “downstream” represent positional relations on the basis of the flow direction from the first stage reaction tank toward the final stage reaction tank.

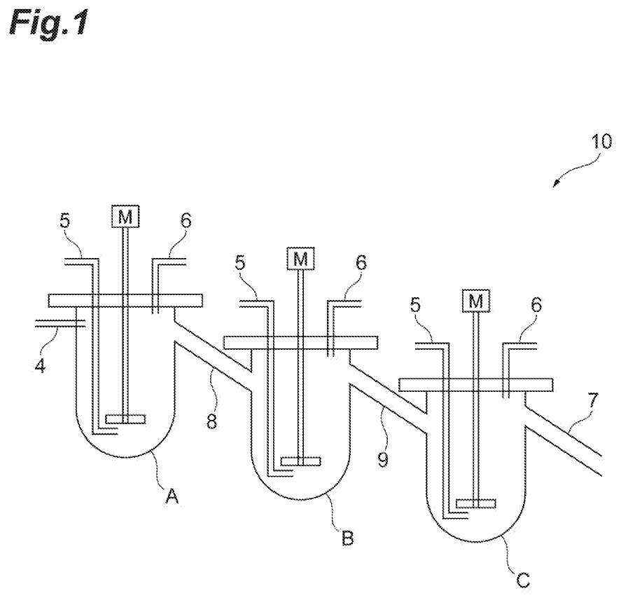

The reactor may be compartmented into a plurality of reaction units. The reactor may be compartmented into a plurality of reaction units (reaction tank) by piping, or may be compartmented into a plurality by inner walls, weirs, or the like. The number of reaction units formed by compartmenting the reactor may be 2 to 5, or may be 2 to 3, from the viewpoint of the control of the reaction and the economic efficiency. It is preferable that the reaction units formed in the reactor are arranged in series along the flow direction of the raw material aqueous solution or the reaction liquid, as shown in FIG. 1. Meanwhile, from the viewpoint of enhancing the processing ability, the reaction units may be arranged in parallel, or reactors arranged in series may be arranged in parallel. The method of installation can be regulated as appropriate according to the purpose of production, the amount of production, the facilities, and the like.

FIG. 1 is a schematic diagram illustrating an example of the reactor. The reactor 10 produces an alkyl nitrite by bringing an aqueous solution containing nitric acid and an alkanol into contact with a gas including nitrogen monoxide. The reactor 10 has three reaction tanks in the manner of being aligned in series. These reaction tanks will be referred to as, from the upstream side, reaction tank A, reaction tank B, and reaction tank C. The reaction tank A and the reaction tank B are connected by a first connection unit 8, and the reaction tank B and the reaction tank C are connected by a second connection unit 9.

The reaction tank A, reaction tank B, and reaction tank C each include a gas supply nozzle 5 and a gas extraction nozzle 6. The reaction tank A is connected to a liquid supply nozzle 4. In the liquid supply nozzle 4, an aqueous solution containing nitric acid and an alkanol is continuously supplied. The aqueous solution supplied to the reaction tank A is brought into contact with a gas including nitrogen monoxide that is continuously supplied through the gas supply nozzle 5.

In the reaction tank A, nitric acid, an alkanol, and nitrogen monoxide react with one another and produce an alkyl nitrite. In this manner, in the reaction tank A, a reaction liquid A including an alkyl nitrite and unreacted nitric acid and unreacted alkanol, and a gas A are obtained. The gas A pulled out through the gas extraction nozzle 6 may include an alkyl nitrite and an alkanol. The nitric acid concentration in the reaction liquid A may be 4% by weight or less, or may be 3% by weight or less.

The reaction liquid A is supplied from the reaction tank A to the reaction tank B via the first connection unit 8. In the reaction tank B, the reaction liquid A is brought into contact with the gas including nitrogen monoxide supplied through the gas supply nozzle 5, and the alkyl nitrite is further produced. Thereby, in the reaction tank B, the reaction liquid B having lower concentrations of nitric acid and the alkanol, and a gas B are obtained from the reaction liquid A. The gas B pulled out through the gas extraction nozzle 6 may include an alkyl nitrite and an alkanol. The nitric acid concentration in the reaction liquid B may be 2% by weight or less, or may be 1% by weight or less.

The reaction liquid B is supplied from the reaction tank B to the reaction tank C via the second connection unit 9. In the reaction tank C, the reaction liquid B is brought into contact with the gas including nitrogen monoxide supplied through the gas supply nozzle 5, and the alkyl nitrite is further produced. Thereby, in the reaction tank C, a reaction liquid C having lower concentrations of alkanol and nitric acid, and a gas C are obtained from the reaction liquid B. The gas C pulled out through the gas extraction nozzle 6 may include an alkyl nitrite and an alkanol. The nitric acid concentration in the reaction liquid C may be 1% by weight or less, or may be 0.5% by weight or less. The reaction liquid C may be treated as an effluent.

As such, the reactor 10 may be compartmented by the first connection unit 8 and the second connection unit 9 to provide a plurality of reaction units, and a first reaction step, a second reaction step, and a third reaction step may be carried out in the reaction tanks A, B, and C. Thereby, it becomes easy to set appropriate reaction conditions according to the nitric acid concentration in the aqueous solution as a raw material. Therefore, the nitric acid concentration can be reduced sufficiently efficiently. The number of the reaction units and the reaction steps is not limited to three, and may be 2 or less, or may be 4 or more.

The alkyl nitrite obtainable by the reaction may be included in the gas A, gas B, gas C, and the reaction liquid C. The alkyl nitrite included in these may be purified by means of a distillation column or the like, or may be used directly in other processes.

A reactor having a plurality of reaction tanks that are arranged in series may include a reaction tank A having a reaction temperature of 60° C. to 80° C., and a reaction tank C having a reaction temperature of 80° C. to 100° C., which is disposed on the downstream side of the reaction tank A. The reaction temperature of the reaction tank A may be 65° C. to 75° C. The reaction temperature of the reaction tank C may be 85° C. to 95° C.

In a case in which the reactor has three or more reaction tanks (reaction units) disposed in series along the flow direction of the aqueous solution or the reaction liquid, it is preferable that the reaction temperature of one or a plurality of reaction tanks B (third reaction unit) other than the reaction tank A (first reaction unit) located on the most upstream side and the reaction tank C (second reaction unit) located on the most downstream side, is higher than or equal to the reaction temperature of the reaction tank A, and lower than or equal to the reaction temperature of the reaction tank C. By aligning a plurality of reaction tanks (reaction units) in series as such, production of alkyl nitrate and the like can be sufficiently suppressed. Therefore, an alkyl nitrite can be more efficiently produced by more effectively utilizing nitric acid. The concentration of alkyl nitrate and the like in the reaction liquid C may be 500 ppm by weight or less, may be 300 ppm by weight or less, or may be 100 ppm by weight or less.

In a reactor (reaction tank) having a nitric acid concentration of 1% by weight or more, the reaction temperature may be set to 60° C. to 80° C., or may be set to 65° C. to 75° C. In a reactor (reaction tank) having a nitric acid concentration of less than 1% by weight, the reaction temperature may be set to 80° C. to 100° C., or may be set to 85° C. to 95° C. Thereby, an alkyl nitrite can be produced more efficiently from nitric acid.

In a case in which a plurality of reaction tanks are disposed in series in the reactor along the flow direction of the aqueous solution or the reaction liquid, the superficial gas velocity at the first reaction unit (reaction tank A) on the most upstream side may be 20 mm/second to 100 mm/second, or may be 30 mm/second to 80 mm/second. The superficial gas velocity at the second reaction unit (reaction tank C) on the most downstream side may be 1 mm/second to 20 mm/second, or may be 5 mm/second or 15 mm/second.

In a case in which three or more reaction units are disposed in series in the reactor, the superficial gas velocity at a reaction unit (for example, reaction tank B) other than the first reaction unit (reaction tank A) disposed on the most upstream side and the second reaction unit (reaction tank C) disposed on the most downstream side, may be higher than or equal to the superficial gas velocity of the second reaction unit and lower than or equal to the superficial gas velocity of the first reaction unit. By employing superficial gas velocities such as described above, an alkyl nitrite can be produced more efficiently by utilizing nitric acid more effectively.

With regard to the reactor (reaction unit) having a nitric acid concentration of 1% by weight or more, the superficial gas velocity may be 20 mm/second to 100 mm/second, or may be 30 mm/second to 80 mm/second. With regard to the reactor (reaction unit) having a nitric acid concentration of less than 1% by weight, the superficial gas velocity may be 1 mm/second to 20 mm/second, or may be 5 mm/second to 15 mm/second. By employing such superficial gas velocities, an alkyl nitrite can be produced more efficiently. The superficial gas velocity according to the present disclosure represents the gas velocity based on a cross-section of the reactor (reaction unit) when the reactor (reaction unit) is assumed to have a tubular shape. Meanwhile, in a case in which the cross-section is not constant, the superficial gas velocity can be determined with an average value of the cross-section.

The retention time for the reactor (reaction unit) may be modified as appropriate according to the reaction conditions and the volume of the reactor (reaction unit). The total retention time of the reactor may be about 1 hour to 20 hours, or may be about 2 hours to 10 hours. The retention time of the reaction liquid at each of the reaction units may vary depending on the reaction temperature and conversion ratio at each of the reaction units; however, from the viewpoints of yield and product quality, the retention time at the reaction unit on the downstream side (second reaction unit) may be set to be longer than the retention time at the reaction unit on the upstream (first reaction unit).

The present disclosure includes the following embodiments.

(1) A method for producing an alkyl nitrite, the method including bringing an aqueous solution containing nitric acid and an alkanol into contact with nitrogen monoxide gas and thereby producing an alkyl nitrite, in which the reaction temperature is 60° C. to 100° C.

(2) The method for producing an alkyl nitrite as described in the item (1), in which the concentration of the alkanol is 50% by weight to 80% by weight with respect to the total amount of the aqueous solution.

(3) The method for producing an alkyl nitrite as described in the item (1) or (2), in which the alkanol is methanol.

(4) The method for producing an alkyl nitrite as described in any one of the items (1) to (3), in which the concentration of nitric acid is 1% by weight to 20% by weight with respect to the total amount of the aqueous solution.

(5) The method for producing an alky nitrite as described in any one of the following items (1) to (4), in which multi-stage reaction tanks of two or more stages arranged in series are used.

(6) The method for producing an alkyl nitrite as described in the item (5), in which when multi-stage reaction tanks are used, the reaction temperature is set to 60° C. to 80° C. at the first stage reaction tank, and the reaction temperature is set to 80° C. to 100° C. at the final stage reaction tank.

(7) The method for producing an alkyl nitrite as described in the item (5) or (6), in which when multi-stage reaction tanks are used, the superficial gas velocity is set to 20 mm/second to 100 mm/second at the first stage reaction tank, and the superficial gas velocity is set to 1 mm/second to 20 mm/second at the final stage reaction tank.

(8) The method for producing an alkyl nitrite as described in any one of the items (1) to (7), in which a transition metal is not included in an amount sufficient for bringing an aqueous solution containing nitric acid and an alkanol into contact with nitrogen monoxide gas and thereby producing an alkyl nitrite.

According to the production method and production apparatus for producing an alkyl nitrite of the various embodiments described above, an alkyl nitrite can be efficiently produced by effectively utilizing nitric acid and the like that are produced as a byproduct in, for example, a method for producing an alkyl nitrite using nitrogen monoxide, oxygen, and an alkanol as starting materials. These production method and production apparatus are capable of sufficiently reducing the nitric acid concentration in the reaction liquid that is finally obtainable, even without using a concentrating column. Therefore, the production method and the production apparatus are particularly suitable for the production of nitric acid esters in an industrial scale.

The nitric acid concentration in the reaction liquid can be decreased to, for example, 1.0% by weight or less. In some other embodiments, a concentrating column may be provided in order to further decrease the nitric acid concentration.

Thus, several embodiments of the present invention have been described; however, the present invention is not intended to be limited to the above-mentioned embodiments. For example, the reactor is not limited to a stirred tank type as shown in FIG. 1. For example, the reactor may be in the form of a multi-stage column including a packed column or trays such as sieve trays. Since the reaction is a gas-liquid contact reaction, it is preferable that the reactor has a stirring function. In the case of using a stirred tank type reactor, from the viewpoint of enhancing stirrability and gas dispersibility, it is preferable to use a reactor having high gas-liquid contact efficiency, which has a blade-shaped stirrer, a rotating apparatus, and the like. In a case in which the reactor is in the form of a multi-stage column, it is preferable that the reactor has a filler material having high gas-liquid contact efficiency. The alkyl nitrite thus produced may be led out of the reaction system while being entrained in gases, (purified by washing or the like as necessary), and then utilized in another reaction.

With regard to the production method and the production apparatus of the present disclosure, the conditions may be changed as appropriate according to the boiling point and solubility of the alkanol, and reactivity. The reaction conditions and the like described above are suitably applied particularly to a method of producing methyl nitrite by bringing an aqueous solution containing nitric acid and methanol into contact with a gas including nitrogen monoxide.

### EXAMPLES

The subject matters of the present invention will be described in more detail by way of Examples and Comparative Examples; however, the present invention is not intended to be limited to the following Examples. Meanwhile, the nitric acid concentration was analyzed by ion chromatography and titration, and others were respectively analyzed by gas chromatography.

### Example 1

A 1-L autoclave equipped with a stirrer (attached with four paddle-type stirring blades), a gas supply nozzle, a gas extraction nozzle, and a liquid draw-off nozzle, was prepared. In a reaction tank, which is such an aerated stirred tank, 800 g of an aqueous solution containing nitric acid and methanol (MeOH) was introduced. The nitric acid concentration in this aqueous solution was 2% by weight, and the methanol concentration was 65% by weight. The reaction tank was purged with nitrogen gas, and then the pressure inside the reaction tank was increased to 0.3 MPaG with nitrogen gas.

Next, while a mixed gas of nitrogen monoxide gas and nitrogen gas (concentration of nitrogen monoxide: 10% by volume) was supplied under stirring (1,100 rpm) through the gas supply nozzle at a rate of 1 NL/h, the temperature of the aqueous solution was increased up to 65° C. The pressure of the reaction tank was maintained at 0.3 MPaG by regulating the amount of gas extracted through the gas extraction nozzle. After the temperature of the aqueous solution had reached 65° C. (reaction temperature), the reaction was continuously carried out for 5 hours under those reaction conditions, and then the nitric acid concentration in the reaction liquid was measured. As a result, the nitric acid concentration was 0.6% by weight. The experiment conditions and results are as shown in Table 1.

The concentration of methyl nitrite in the gas extracted through the gas extraction nozzle of the reaction tank was 10% by volume.

### Example 2

The production was carried out in the same manner as in Example 1, except that the reaction temperature was changed to 75° C. The experiment conditions and results are as shown in Table 1. The concentration of methyl nitrite in the gas extracted through the gas extraction nozzle was 10% by volume.

### Example 3

The production was carried out in the same manner as in Example 1, except that the reaction temperature was changed to 85° C.

The experiment conditions and results are as shown in Table 1. The concentration of methyl nitrite in the gas extracted through the gas extraction nozzle was 10% by volume.

### Example 4

The production was carried out in the same manner as in Example 1, except that the reaction temperature was changed to 95° C. The experiment conditions and results are as shown in Table 1. The concentration of methyl nitrite in the gas extracted through the gas extraction nozzle was 10% by volume.

### Comparative Example 1

The production was carried out in the same manner as in Example 1, except that the reaction temperature was changed to 50° C. The experiment conditions and results are as shown in Table 1. The concentration of methyl nitrite in the gas extracted through the gas extraction nozzle was 10% by volume.

### Example 5

The production was carried out in the same manner as in Example 1, except that the methanol concentration was changed to 55% by weight. The experiment conditions and results are as shown in Table 1. The concentration of methyl nitrite in the gas extracted through the gas extraction nozzle was 9% by volume.

### Example 6

The production was carried out in the same manner as in Example 1, except that the methanol concentration was changed to 75% by weight. The experiment conditions and results are as shown in Table 1. The concentration of methyl nitrite in the gas extracted through the gas extraction nozzle was 11% by volume.

### Example 7

The production was carried out in the same manner as in Example 1, except that the methanol concentration was changed to 45% by weight. The experiment conditions and results are as shown in

Table 1. The concentration of methyl nitrite in the gas extracted through the gas extraction nozzle was 8% by volume.

TABLE 1

| | | | Methanol | Nitric acid | | | |
|---|---|---|---|---|---|---|---|
| | | | concentration | concentration | | | Nitric acid |
| | | | in aqueous | in aqueous | Feed gas | Reaction | concentration |
| Experiment | Reaction | Temperature | solution | solution | amount | time | after reaction |
| No. | tank | ° C. | wt % | wt % | NL/h | Hours | wt % |
| Example 1 | Aerated | 65 | 65 | 2.0 | 1 | 5 | 0.6 |
| | stirred tank | | | | | | |
| Example 2 | Same as | 75 | 65 | 2.0 | 1 | 5 | 0.4 |
| | above | | | | | | |
| Example 3 | Same as | 85 | 65 | 2.0 | 1 | 5 | 0.1 |
| | above | | | | | | |
| Example 4 | Same as | 95 | 65 | 2.0 | 1 | 5 | 0.05 |
| | above | | | | | | |
| Comparative | Same as | 50 | 65 | 2.0 | 1 | 5 | 1.5 |
| Example 1 | above | | | | | | |
| Example 5 | Same as | 65 | 55 | 2.0 | 1 | 5 | 1.0 |
| | above | | | | | | |
| Example 6 | Same as | 65 | 75 | 2.0 | 1 | 5 | 0.3 |
| | above | | | | | | |
| Example 7 | Same as | 65 | 45 | 2.0 | 1 | 5 | 1.4 |
| | above | | | | | | |

### Example 8

As shown in FIG. 1, three 1-L autoclaves each equipped with a stirrer (attached with four paddle-type stirring blades), a liquid supply nozzle, a gas supply nozzle, a gas extraction nozzle, a hot water jacket for temperature increase, and an overflow-induced liquid draw-off nozzle, were connected in series. Methyl nitrite was produced by the following procedure using a reactor including three aerated stirred tank as such. Specifically, 800 g of an aqueous solution containing nitric acid and methanol was introduced into each of the three aerated stirred tanks (reaction tank A, reaction tank B, and reaction tank C). The nitric acid concentration in each of the aqueous solutions was 6% by weight, and the methanol concentration was 65% by weight. The respective reaction tanks were purged with nitrogen gas, and then the pressure inside the reaction tanks was increased to 0.3 MPaG with nitrogen gas. After this, the pressure in the respective reaction tanks was maintained at 0.3 MPaG by regulating the extraction amount of gas through the gas extraction nozzle.

Next, under stirring (1,100 rpm), a mixed gas of nitrogen monoxide gas and nitrogen gas (concentration of nitrogen monoxide: 10% by volume) was supplied to the respective reaction tanks through the respective gas supply nozzles. While the mixed gas was supplied such that the superficial gas velocity at the reaction tank A would be 60 mm/second, the superficial gas velocity at the reaction tank B would be 15 mm/second, and the superficial gas velocity at the reaction tank C would be 10 mm/second, the temperature of the reaction tank A was increased to 70° C., the temperature of the reaction tank B was increased to 80° C., and the temperature of the reaction tank C was increased to 90° C. Subsequently, the respective reaction tanks were maintained at these temperatures (reaction temperatures).

After the temperature increase, an aqueous solution containing nitric acid and methanol was continuously supplied at a rate of 500 g/hour through the liquid supply nozzle of the reaction tank A. The nitric acid concentration in the aqueous solution was 6% by weight, and the methanol concentration was 65% by weight. When the solution in the reaction tank A reached a predetermined amount or more, the solution passed sequentially from the reaction tank A to a first connection unit, the reaction tank B, a second connection unit, and the reaction tank C, and then the solution was discharged through the liquid discharge nozzle of the reaction tank C. In this manner, the reaction was continuously carried out while the liquid surfaces of the reaction tank A, reaction tank B, and reaction tank C were maintained constant.

After the reaction in the respective reaction tanks had been stabilized, the nitric acid concentration and the concentration of methyl nitrate in the reaction liquid (effluent) discharged through the liquid draw-off nozzle of the reaction tank C were measured. As a result, the nitric acid concentration was 0.1% by weight, and the methyl nitrate concentration was 10 ppm by weight or less. Furthermore, the nitric acid concentrations in the solutions (reaction liquid A and reaction liquid B) at the first connection unit and the second connection unit were measured. As a result, the nitric acid concentration at the first connection unit (reaction liquid A) was 2.2% by weight, and the nitric acid concentration at the second connection unit (reaction liquid B) was 0.8% by weight. As such, it was verified that the nitric acid concentration in the solution was decreased stepwise.

The concentration of methyl nitrite in the confluent gas obtained through confluence of the gases extracted through the gas extraction nozzles of the respective reaction tanks was 9.6% by volume. The experiment conditions and results are as shown in Table 2.

### Example 9

The production was carried out in the same manner as in Example 8, except that the reaction temperatures of the reaction tank A, reaction tank B, and reaction tank C were all changed to 70° C. The results are as shown in Table 2.

### Example 10

The production was carried out in the same manner as in Example 8, except that the reaction temperatures of the reaction tank A, reaction tank B, and reaction tank C were all changed to 90° C. The results are as shown in Table 2.

TABLE 2

| | | | Nitric acid | | |
|---|---|---|---|---|---|
| | | | concentration in | Nitric acid | Methyl nitrate |
| | | | aqueous | concentration in | concentration in |
| | | | solution | reaction liquid | reaction liquid |
| No. | Reactor | Reaction temperature | wt % | wt % | wt ppm |
| Example 8 | Aerated | Reaction tank A 70° C. | 6 | 0.1 | <10 |
| | stirred tank | Reaction tank B 80° C. | | | |
| | (3 stages) | Reaction tank C 90° C. | | | |
| Example 9 | Aerated | Reaction tank A 70° C. | 6 | 0.7 | 30 |
| | stirred tank | Reaction tank B 70° C. | | | |
| | (3 stages) | Reaction tank C 70° C. | | | |
| Example 10 | Aerated | Reaction tank A 90° C. | 6 | 0.1 | 200 |
| | stirred tank | Reaction tank B 90° C. | | | |
| | (3 stages) | Reaction tank C 90° C. | | | |

### INDUSTRIAL APPLICABILITY

According to the production method and production apparatus for producing an alkyl nitrite of the present disclosure, the nitric acid concentration in the effluent can be sufficiently decreased even without using a concentrating column. Furthermore, an industrially suitable production method and an industrially suitable production apparatus for producing an alkyl nitrite, by which an alkyl nitrite can be efficiently produced, can be provided. Alkyl nitrites are compounds useful for various oxidation processes (production of dialkyl oxalates, dialkyl carbonates, and the like).

### REFERENCE SIGNS LIST

A, B, C: reaction tank, 4: liquid supply nozzle, 5: gas supply nozzle, 6: gas extraction nozzle, 7: liquid draw-off nozzle, 8: first connection unit, 9: second connection unit, 10: reactor, M: stirrer.

1. A method for producing an alkyl nitrite, the method comprising:
bringing an aqueous solution containing nitric acid and an alkanol into contact with a gas including nitrogen monoxide and thereby producing an alkyl nitrite, wherein a reaction temperature is 60° C. to 100° C.

2. The method for producing an alkyl nitrite according to claim 1, wherein a concentration of the alkanol is 50% by weight to 80% by weight with respect to the total amount of the aqueous solution.

3. The method for producing an alkyl nitrite according to claim 1, wherein a concentration of the alkanol is 60% by weight or more with respect to the total amount of the aqueous solution.

4. The method for producing an alkyl nitrite according to claim 1, wherein a concentration of nitric acid in a reaction liquid obtainable by bringing the aqueous solution into contact with the gas is decreased to 1% by weight or less.

5. The method for producing an alkyl nitrite according to claim 1, wherein the alkanol includes methanol.

6. The method for producing an alkyl nitrite according to claim 1, wherein a concentration of nitric acid in the aqueous solution is 1% by weight to 20% by weight with respect to the total amount of the aqueous solution.

7. The method for producing an alkyl nitrite according to claim 1, the method comprising:
a first reaction step of producing the alkyl nitrite at a reaction temperature of 60° C. to 80° C.; and
a second reaction step of producing the alkyl nitrite at a reaction temperature of 80° C. to 100° C.

8. The method for producing an alkyl nitrite according to claim 7, wherein a superficial gas velocity in the first reaction step is 20 mm/second to 100 mm/second, and a superficial gas velocity in the second reaction step is 1 mm/second to 20 mm/second.

9. The method for producing an alkyl nitrite according to claim 1, wherein the alkyl nitrite is produced using a reactor compartmented into two or more chambers along a flow direction of the aqueous solution.

10. The method for producing an alkyl nitrite according to claim 9, wherein the reactor has a first reaction unit operated in a temperature range of 60° C. to 80° C., and a second reaction unit disposed on a downstream side of the first reaction unit and operated in a temperature range of 80° C. to 100° C.

11. The method for producing an alkyl nitrite according to claim 10, wherein a superficial gas velocity at the first reaction unit is 20 mm/second to 100 mm/second, and a superficial gas velocity at the second reaction unit is 1 mm/second to 20 mm/second.

12. The method for producing an alkyl nitrite according to claim 10, wherein the first reaction unit is located on the most upstream side of the reactor, and the second reaction unit is located on the most downstream side of the reactor.

13. The method for producing an alkyl nitrite according to claim 1, wherein the use amount of a catalyst containing a transition metal with respect to the aqueous solution is less than 0.1% by weight in terms of the transition metal.

14. An apparatus for producing an alkyl nitrite, the apparatus comprising:
a reactor for bringing an aqueous solution containing nitric acid and an alkanol into contact with a gas including nitrogen monoxide and thereby producing an alkyl nitrite,
wherein a reaction temperature in the reactor is 60° C. to 100° C.

15. The apparatus for producing an alkyl nitrite according to claim 14, wherein the reactor has two or more reaction units compartmented along a flow direction of the aqueous solution.

16. The apparatus for producing an alkyl nitrite according to claim 14, wherein the reactor has a first reaction unit, and a second reaction unit located on a downstream side of the first reaction unit, the second reaction unit producing the alkyl nitrite at a reaction temperature higher than the reaction temperature of the first reaction unit.

17. The apparatus for producing an alkyl nitrite according to claim 14, wherein in the reactor, a nitric acid concentration in a reaction liquid is decreased to 1% by weight or less.

18. The method for producing an alkyl nitrite according to claim 1, wherein the alkyl nitrite is produced using a reactor compartmented into 2 to 5 chambers along a flow direction of the aqueous solution.

19. The method for producing an alkyl nitrite according to claim 1, nitrogen monoxide is used at a proportion of 1 mol to 50 mol with respect to 1 mol of nitric acid.

20. The apparatus for producing an alkyl nitrite according to claim 14, wherein the reactor has no nitric acid concentrating column.
